(19) United States
(12) Patent Application Publication
(10) Pub. No.: US 2010/0275932 A1
(43) Pub. Date: Nov. 4, 2010

(54) CHILD SWADDLE APPARATUS

(76) Inventor: SARAH SHACKLETON, Los Angeles, CA (US)

Correspondence Address: COHEN I.P. LAW GROUP, P.C., 9025 Wilshire Blvd., Suite 301, Beverly Hills, CA, 90211, US

(21) Appl. No.: 12/610,262

(22) Filed: Oct. 30, 2009

Related U.S. Application Data
(60) Provisional application No. 61/109,769, filed on Oct. 30, 2008.

(51) Int. Cl. A61F 5/37; A41B 13/06

(52) U.S. Cl. 128/872

(57) ABSTRACT

A swaddle apparatus for wrapping around arms and upper torso of a child while changing a diaper is disclosed. The child swaddle apparatus comprises a fitted sheet, a traverse main body panel with left and right extensions on opposite sides and a swaddle means attached to the left and right extensions. The main body panel is affixed onto the fitted sheet so that the left and right extensions may extend over the opposite ends of the fitted sheet. The child with shoulders is placed above the upper end of the fabric panel. The arms of the child are restrained close and parallel to the upper torso and the right extension is wrapped over the child's arms and upper torso. The left extension is folded over the right extension and fastened using the swaddle means thereby restricting the movement of the child and keeping the child warm, safe, comfortable, and calm.

### CROSS-REFERENCE TO RELATED APPLICATIONS

This application claims the benefit of PPA Ser. No. 61/109,769, filed Oct. 30, 2008 by the present inventor.

### STATEMENT REGARDING FEDERALLY SPONSORED RESEARCH AND DEVELOPMENT

Not Applicable.

### FIELD OF THE INVENTION

This invention relates to a child swaddle apparatus, and more particularly to an apparatus to be used with a child during a diaper change.

### DISCUSSION OF RELATED ART

Changing an infant's diaper can often be a difficult task for parents and babysitters since it typically involves placing the child on an elevated, level surface such as a changing table with a foam changing pad; removing the outer garments to expose the soiled diaper; removing the soiled diaper; replacing the old diaper with a fresh diaper; and then redressing the child. There is also the discomfort that comes with the soiled diaper, the isolation from the parent or guardian as the child is laid on the changing pad, along with the chill that comes from being wet and suddenly snaked. This may cause the child to cry, flail its arms and legs, and become upset, which delays the diaper change and may cause the care-giver and the baby unnecessary stress. Moreover, the Moro reflex, which is normally present at birth through the age of six months old, is frequently exhibited by babies during a diaper change. This reflex can make the diaper change more difficult and time consuming, as well as potentially dangerous if the baby squirms off the changing pad or flails its limbs against neighbouring structures such as walls, changing table corners, etc.

Keeping a baby's temperature stable is an important consideration when changing a diaper. A baby's body surface is about three times greater than an adult's, compared to the weight of his/her body. Babies can lose heat rapidly, as much as four times more quickly than adults. Premature and low birth weight babies usually have little body fat and may be too immature to regulate their own temperature, even in a warm environment. Even full-term and healthy newborns may not be able to maintain their body temperature if the environment is too cold. When babies are cold-stressed, they use energy and oxygen to generate warmth. If skin temperatures drop just one degree from the ideal, a baby's oxygen use can increase by 10 percent. By keeping babies at optimal temperatures, neither too hot or cold, they can conserve energy and build up reserves. This is especially important when babies are sick or premature.

One prior art device, in U.S. Pat. No. 6,009,874 issued to Sartin on Jan. 4, 2000, discloses a diaper changing restraint system for restraining an infant while changing the infant's diaper. The system provides a four-point restraint system for restraining the infant by itself or in conjunction with a diaper-changing table. The system includes a bottom portion, a top portion and a mechanism for fastening the top portion to the bottom portion. A fastening mechanism, preferably hook and pile is provided for attaching the top portion to the bottom portion via the two shoulder straps and the two side portions. In use, the stiff stable surface of the bottom portion is positioned under the pad of a typical diaper-changing table with the four cloth-like portions extending there from. The infant is placed on the pad, and the top portion is then placed over the infant. The four-point attachment scheme may be tedious and uncomfortable for caregivers and the baby after repeated use. Furthermore, repeated removal and reattachment of the four point restraint system from the base structure increases the likelihood that the restraint system will be lost or misplaced away from the changing area.

U.S. Pat. No. 6,755,198 issued to Parker provides a system for restraining the upper torso of a child or infant for diapering activities. The system provides a stable base onto which the child is laid, and provides restraining straps which are attached to the base and arranged over the chest of the child in a crisscrossing manner. The child is prevented from rolling, sitting and sliding by the combined action of the restraining straps and the stable base. A significant disadvantage of this device is that the system fails to adequately restraint the infant. For instance, the infant's hands are free to manipulate the straps, and thus potentially slip out of the device.

### SUMMARY OF THE INVENTION

The present invention is a swaddle apparatus for wrapping around arms and upper torso of a child while changing a diaper. The child swaddle apparatus comprises a fitted sheet having at least one trim at each corner, a traverse main body panel having a front surface, a rear surface left and right extensions bordering the main body panel on opposite sides and a swaddle means attached to the left and right extensions. The mid-portion of the rear surface of the main body panel is affixed onto a central portion of the fitted sheet so that the left and right extensions may hang over the opposite ends of the fitted sheet. The swaddle means disposed on the left and right extensions may comprise hook and loop components. The front surface may include a fabric panel at a central portion and a plurality of darts proximate to an upper end of the fabric panel.

In use, a child may be placed with shoulders above the upper end of the fabric panel. The main body panel may fall just above the child's umbilicus to allow for the diaper to be changed. The left and right extensions may also incorporate a flap (not shown) to cover the swaddle means and a pocket to slid the arms of the child and to assist in restraining the child. The arms of the child is restrained close and parallel to the upper torso and then the right extension of the main body panel is wrapped over the child's arms and upper torso. The left extension is folded over the right extension to enclose the child firmly and the left and right extensions over the child is fastened using the swaddle means thereby restricting the movement of the child and keeping the child warm, safe, comfortable, and calm.

The present invention addresses the issues of the diaper change and unstable thermoregulatory capabilities of infants discussed above by introducing a changing pad cover with fastening wings that can wrap around a child during the diaper change to swaddle the child while restricting its movement and exposing the lower torso, thereby keeping the baby warm, safe, comfortable, and calm.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

FIG. 1 is a perspective view of a preferred embodiment of a child swaddle apparatus in accordance with the present invention;

FIG. 2 is a perspective view of the preferred embodiment of the child swaddle apparatus in accordance with the present invention illustrating a child lying thereon;

FIG. 3 is a front plan view of the preferred embodiment in accordance with the present invention illustrating the child being partially swaddled by a right extension of the child swaddle apparatus;

FIG. 4 is a front plan view of the preferred embodiment in accordance with the present invention illustrating the child being fully swaddled by left and right extensions of the child swaddle apparatus; and

FIG. 5 is a front plan view of an alternative embodiment in accordance with the present invention illustrating the child lying thereon.

### DETAILED DESCRIPTION OF THE PREFERRED EMBODIMENT

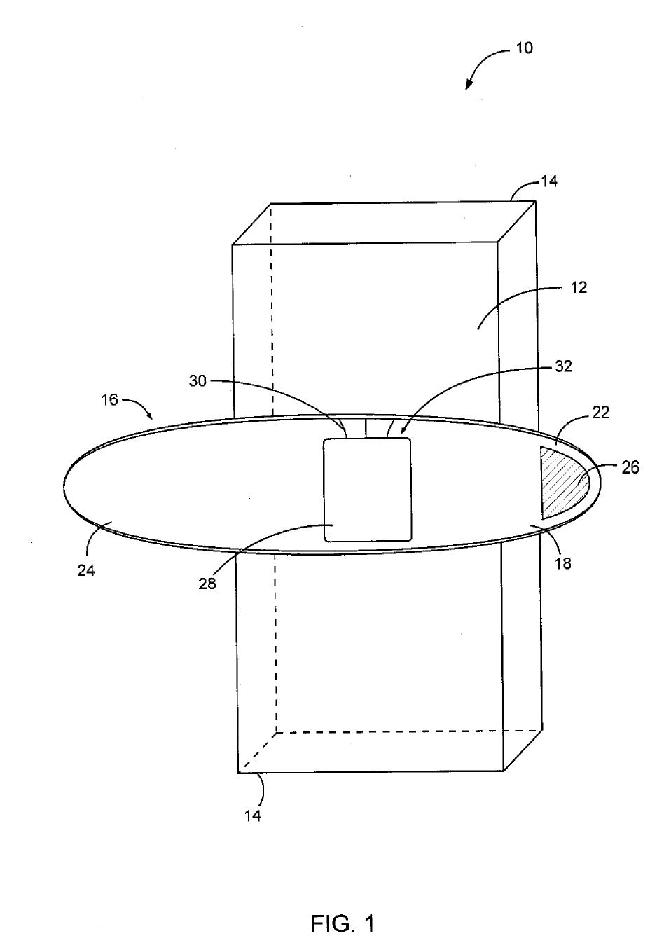

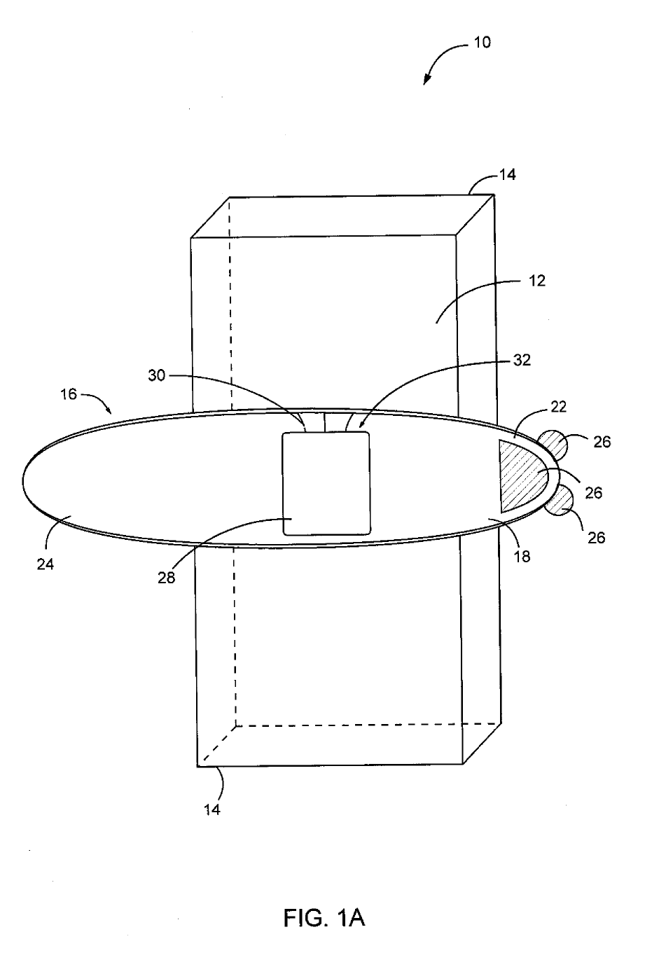

FIG. 1 is a perspective view of a preferred embodiment in accordance with the present invention illustrating a swaddle apparatus 10 for wrapping around arms and upper torso of a child while changing a diaper. The child swaddle apparatus 10 comprises a fitted sheet 12 having at least one trim 14 at each corner, a traverse main body panel 16 having a front surface 18, a rear surface (not shown), left and right extensions 22, 24 bordering the main body panel 16 on opposite sides (not shown) and a swaddle means 26 attached to the left and right extensions 22, 24. One swaddle means 26 is attached to the inner side of left extension 22 while another swaddle means is attached to the outer side of the right extension 24. Additional swaddle means may be attached to the outer portion the left extension 22 as show in FIG. 1A. The fitted sheet 12 is sized to be placed over a changing mat and may incorporate a soft and comforting washable fabric such as chamois polyester. The mid-portion of the rear surface of the main body panel (not shown) is affixed onto a central portion of the fitted sheet (not shown) so that the left and right extensions 22, 24 may hang over the opposite ends of the fitted sheet 12. The swaddle means 26 disposed on the left and right extensions 22, 24 may comprise hook and loop components. The front surface 18 may include a fabric panel 28 at a central portion (not shown) and also a plurality of darts 30 proximate to an upper end of the fabric panel 32.

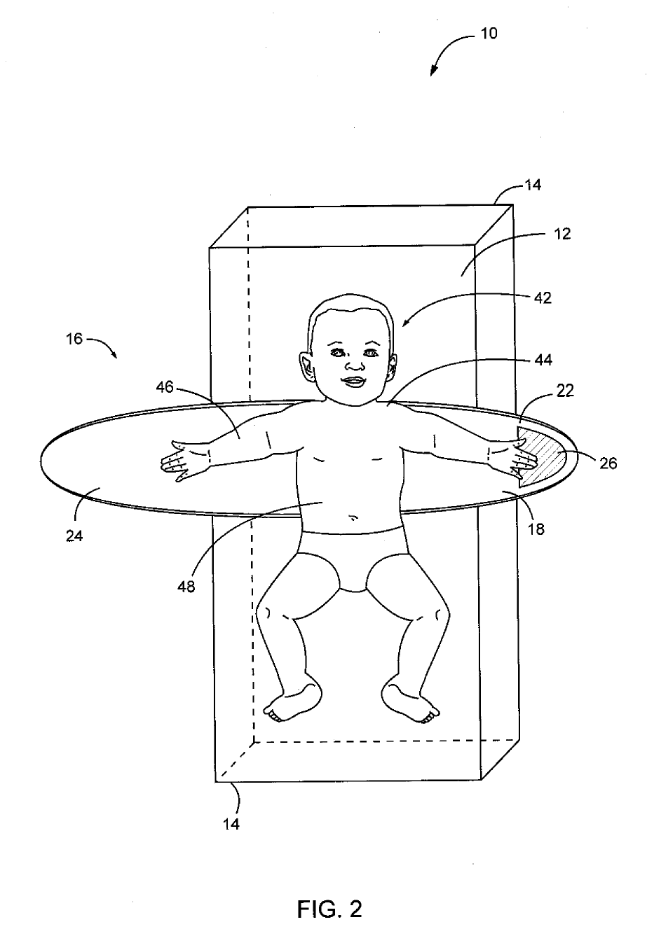

FIG. 2 is a perspective view of the preferred embodiment in accordance with the present invention illustrating a child 42 placed with shoulders 44 above the upper end of the fabric panel 32. The main body panel 16 may fall just above the child's umbilicus and around upper torso 48 to allow for the diaper to be changed. The left and right extensions 22, 24 may also incorporate a flap (not shown) to cover the swaddle means 26 and a pocket (not shown) to slid the arms of the child 46 and to assist in restraining the child 42. The pockets may be formed by sewing a portion of material over the opposite ends of the extensions (not shown) with an opening to allow for ingress and egress of the child's hands.

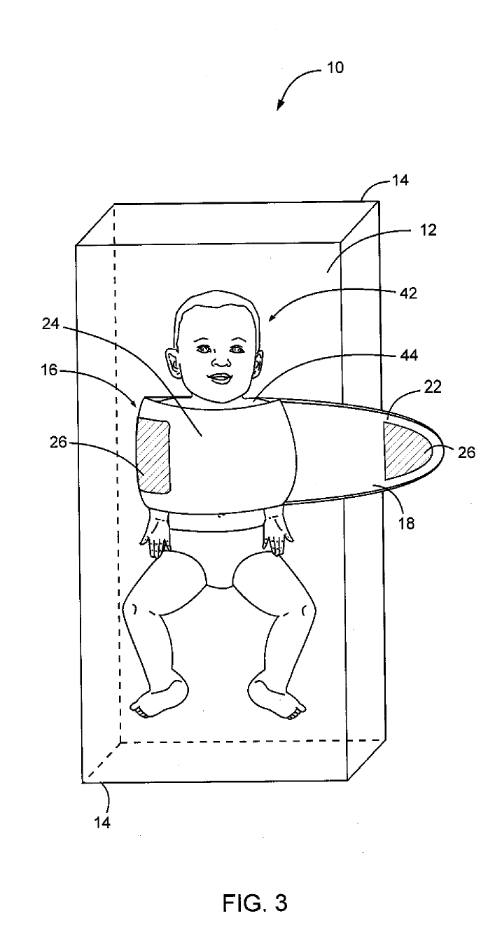

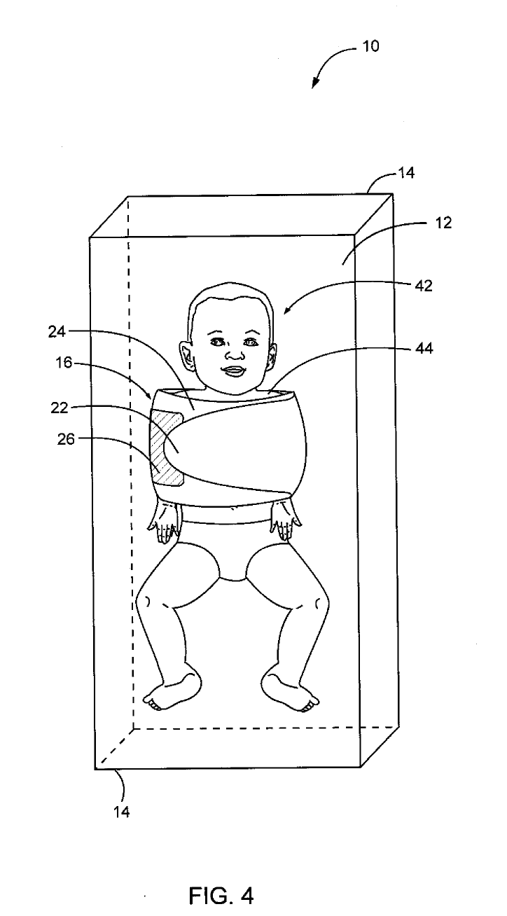

FIGS. 3 and 4 illustrate a method of using the swaddle apparatus 10 while changing the diaper of the child 42. As a first step, place the child 42 upon the fabric panel of the main body panel 28 with the arms 46 and upper torso 48 of the child 42 thereon as in FIG. 2. After placing the child 42 upon the fabric panel of the main body panel 28, restrain the arms of the child 46 close and parallel to the upper torso 48 and then wrap the right extension of the main body panel 24 over the child's arms 46 and upper torso 48 as shown in FIG. 3. Then, as illustrated in FIG. 4, fold the left extension 22 over the right extension 24 to enclose the child 42 firmly and fasten the left and right extensions 22, 24 over the child 42 using the swaddle means 26 thereby restricting the movement of the child and keeping the child warm, safe, comfortable, and calm.

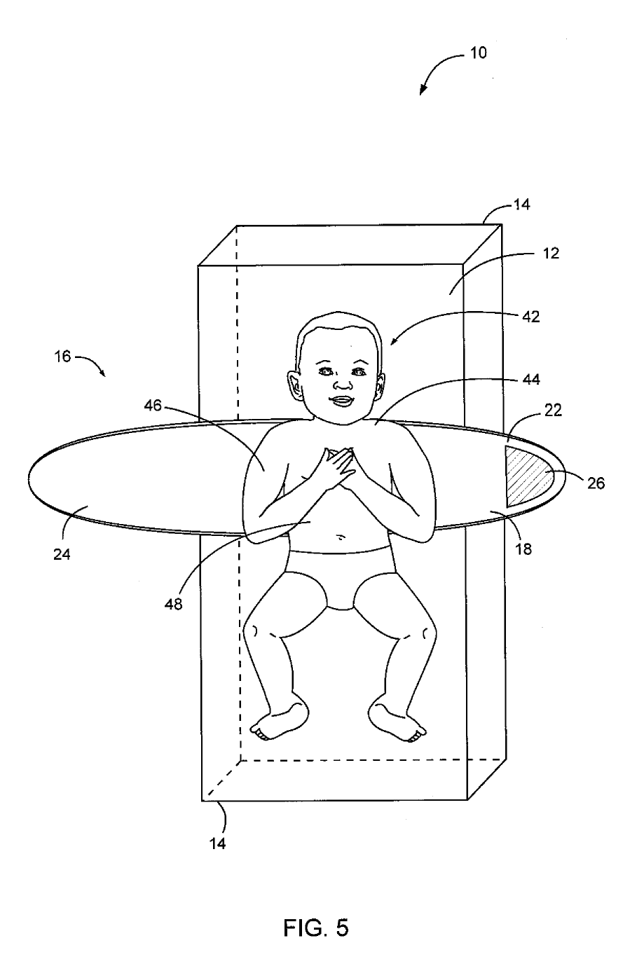

FIG. 5 illustrates an alternate method of using the swaddle apparatus 10 while changing the diaper of the child 42. In this method the child 42 is placed upon the fabric panel of the main body panel 28 with the arms 46 and upper torso 48 of the child 42 thereon as in FIG. 2. After placing the child 42 upon the fabric panel of the main body panel 28, the arms of the child 46 are restrained by placing the hands of the child on the upper torso 48 with the elbows of said arms 46 bent. Then wrap the right extension of the main body panel 24 over the child's arms 46 and upper torso 48 as shown in FIG. 3. Then, as illustrated in FIG. 4, fold the left extension 22 over the right extension 24 to enclose the child 42 firmly and fasten the left and right extensions 22, 24 over the child 42 using the swaddle means 26 thereby restricting the movement of the child and keeping the child warm, safe, comfortable, and calm.

A further embodiment of the present invention uses the swaddle apparatus 10 for older or larger babies in which the arm of the child 42 are placed outside or above the left and right extensions 22 and 24. The right and left extensions 22 and 24 are slightly narrower in width such the top of the extensions 22 and 42 are under the armpits of the child 24.

While a particular form of the invention has been illustrated and described, it will be apparent that various modifications can be made without departing from the spirit and scope of the invention. Accordingly, it is not intended that the invention be limited, except as by the appended claims.

What is claimed is:

1. A child swaddle apparatus for wrapping around at least one child's arms and upper torso while changing a diaper comprising.
a fitted sheet having at least one trim at each corner thereon;
a traverse main body panel having a front surface, a rear surface and left and right extensions bordering the main body panel on opposite sides thereof, wherein a mid-portion of the rear surface being affixed onto a central portion of the fitted sheet; and
a swaddle means attached to the left and right extensions;
whereby the left and right extensions of the traverse main body panel fold around the at least one child's arms and upper torso to restrict the movement of the at least one child thereby keeping the at least one child warm, safe, comfortable and calm.

2. The child swaddle apparatus of claim 1 wherein the main body panel includes a fabric panel at a central portion of the front surface.

3. The child swaddle apparatus of claim 1 wherein the front surface includes a plurality of darts proximate to an upper end of the fabric panel.

4. The child swaddle apparatus of claim 1 wherein the left and right extensions may be of width and length sufficient to overlap an infant.

5. The child swaddle apparatus of claim 1 wherein the swaddle means comprises shook and loop components.

6. The child swaddle apparatus of claim 1 wherein the swaddle means is arranged to releasably restrain the left and right extensions to each other when overlapped about the at least one child.

7. The child swaddle apparatus of claim 1 wherein the left and right extensions may incorporate a flap to cover the swaddle means.

8. The child swaddle apparatus of claim 1 wherein the left and right extensions may also incorporate a pocket on at least one end to slid the arms of the at least one child.

9. The child swaddle apparatus of claim 1 wherein the main body panel may be made from any pliable material.

10. The child swaddle apparatus of claim 1 wherein the trim of the fitted sheet may be of elastic, plastic or the like.

11. A method of using a swaddle apparatus while changing a diaper of at least one child comprising the steps of
(a) providing the swaddle apparatus having a fitted sheet and a main body panel;
(b) laying the at least one child upon a fabric panel of the main body panel with at least one child's arm's and upper torso thereon;
(c) restraining the at least one child's arms close and parallel to the at least one child's torso;
(d) wrapping a right extension of the main body panel over the at least one child's arms and upper torso;
(e) folding over a left extension over the right extension to enclose the at least one child firmly; and
(f) fastening the left and right extensions over the at least one child using at least one swaddle means.

13. The method of claim 11 wherein the fabric panel is arranged to a center portion of a front surface of the main body panel.

14. The method of claim 11 wherein the left and right extensions may be of width sufficient to overlap the at least one child.

15. The method of claim 11 wherein the swaddle means comprises hook and loop components.

16. A method of using a swaddle apparatus while changing a diaper of at least one child comprising the steps of:
(a) providing the swaddle apparatus having a fitted sheet and a main body panel;
(b) laying the at least one child upon a fabric panel of the main body panel with at least one child's arm's and upper torso thereon;
(c) restraining the at least one child's arms by placing the child's hands on the upper torso with the elbows bent;
(d) wrapping a right extension of the main body panel over the at least one child's arms and upper torso;
(e) folding over a left extension over the right extension to enclose the at least one child firmly; and
(f) fastening the left and right extensions over the at least one child using at least one swaddle means.

17. The method of claim 16 wherein the fabric panel is arranged to a center portion of a front surface of the main body panel.

18. The method of claim 16 wherein the left and right extensions may be of width sufficient to overlap the at least one child.

19. The method of claim 16 wherein the swaddle means comprises hook and loop components.
